SEQUENCE LISTING (10 sequences)

SEQ ID NO: 1 - protein, 102 aa, artificial
  polypeptide
VSDVPRDLEVVAATPTSLLISWHHLSCSDSRYYRITYGETGGNSPVQEFTVPGSYDSYSATISGLKPGVDYTATVYAVTRRGYDCPHPISINYRTEIDKPSR

SEQ ID NO: 2 - protein, 98 aa, artificial
  polypeptide
ASDVPRDLEVIAATPTSLLISWLPSYSPGYYRITYGETGGNSPVQEFTVPGSYDSYSATISGLKPGVDYTITVYAVTGRVSSYPTSINCRTGIDKPSR

SEQ ID NO: 3 - protein, 98 aa, artificial
  polypeptide
SSDSPRNLEVTNATPNSLTISWDDPYPAHAHYYRITYGETGGNSSSQEFTVPGNYYYATISGLKPGQDYTITVYAVGTNSYSNPISINYRTETDKPSQ

SEQ ID NO: 4 - protein, 99 aa, artificial
  polypeptide
SPDSPRNLEVTNATPNSLTISWDDPYPAHAHYYRITYGETGGNSSSQEFTVPGNYYYATISGLKPGQDYTITVYAVGCKNCYSNPISINYRTEIDRPSQ

SEQ ID NO: 5 - protein, 99 aa, artificial
  polypeptide
SSDSPRNLEVTNATPNSLTISWDDPYPTHAHYYRITYGETGGNSSSQEFTVPGNYYYATISGLKPGQDYTITVYAVGSHGHYSNPTSINYRTEIDKPSQ

SEQ ID NO: 6 - protein, 100 aa, artificial
  polypeptide
SSDSPRNLEVTNATPNSLTISWDDPYPAHAHYYRITYGETGGNSSSQEFTVPGNYYYATISGLKPGQDYTITVYAVTDHNYSYSNPISINCRTEIDKPSQ

SEQ ID NO: 7 - protein, 99 aa, artificial
  polypeptide
SSDSPRNLEVTNATPNSLTVSWDDPYPTHAHYYRITYGETGGSSSSQEFTVPGNYYYATISGLKPGQDYTITVYAVSNDNSSSNPISINYRTEIDKPSQ

SEQ ID NO: 8 - protein, 88 aa, artificial
  polypeptide
SSDSPRNLEVTNATPNSLTISWDAPPALDSSGYRITYSETGGNSPSQEFTVPGNYYYATISRLKPGQDYTITVYTVSHHDRYGPYSNP

SEQ ID NO: 9 - protein, 99 aa, artificial
  polypeptide
SSDSPRDLEVTNATPNSLTISWDAPPALDSSGYRITYGETGGNSPSQEFTVPGNYYYATISGLKPGQDYTITVYAVADNSHNPNPISINYRTEIDKPSQ

SEQ ID NO: 10 - protein, 98 aa, artificial
  polypeptide
SSDSPRNLEVINATPNSLTISWDAPPALDSSGYRITYGETGGNSPSQEFTVPGNYYYATISGLRPGQDYTITVYAVSNIDNSNPVSINYRTEIDKPSQ

(19) United States
(12) Patent Application Publication
(10) Pub. No.: US 2017/0002058 A1
(43) Pub. Date: Jan. 5, 2017

(54) COMPOSITIONS AND METHODS THAT INVOLVE PROTEIN SCAFFOLDS THAT SPECIFICALLY BIND TO HEPATOCYTE GROWTH FACTOR RECEPTOR

(71) Applicant: REGENTS OF THE UNIVERSITY OF MINNESOTA, Minneapolis, MN (US)

(72) Inventors: Benjamin Joseph Hackel, Edina, MN (US); Daniel Ray Woldring, Minneapolis, MN (US); Hong Zhou, Minneapolis, MN (US)

(21) Appl. No.: 15/198,839

(22) Filed: Jun. 30, 2016

Related U.S. Application Data
(60) Provisional application No. 62/186,498, filed on Jun. 30, 2015.

(51) Int. Cl. C07K 14/78; G01N 33/573

(52) U.S. Cl. CPC C07K 14/78; G01N 33/573; A61K 38/00

(57) ABSTRACT

This disclosure describes a non-naturally occurring protein scaffold that specifically binds to hepatocyte growth factor receptor (MET) and methods of using such protein scaffolds. Generally, the protein scaffold includes a frame and at least one loop region that specifically binds hepatocyte growth factor receptor (MET). The frame generally includes a plurality of structural domains that include at least one β structure or at least one a helix. The loop region generally includes an amino acid sequence that varies from a naturally-occurring loop region by at least one amino acid deletion, amino acid substitution, or amino acid addition.

### CROSS-REFERENCE TO RELATED APPLICATION

This application claims priority to U.S. Provisional Patent Application No. 62/186,498, filed Jun. 30, 2015, which is incorporated herein by reference.

### GOVERNMENT FUNDING

This invention was made with government support under UL1TR000114 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “11004740101_SequenceListing_ST25.txt” having a size of 10 KB and created on Jun. 30, 2016. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY

This disclosure describes, in one aspect, a non-naturally occurring protein scaffold that specifically binds to hepatocyte growth factor receptor (MET). Generally, the protein scaffold includes a frame and at least one loop region that specifically binds hepatocyte growth factor receptor (MET). The frame generally includes a plurality of structural domains that include at least one β structure or at least one a helix. The loop region generally includes an amino acid sequence that varies from a naturally-occurring loop region by at least one amino acid deletion, amino acid substitution, or amino acid addition.

In some embodiments, the frame is derived from fibronectin.

In some embodiments, a loop region can include at least one of SEQ ID NO:1-10.

In some embodiments, the protein scaffold may be formulated into a pharmaceutical composition.

In some embodiments, the protein scaffold may be formulated into a detection composition. In some of these embodiments, the protein scaffold composition can further include a detectable marker. In some of these embodiments, the detectable marker can include a radioactive isotope, a fluorescent marker, or a colorimetric marker.

In another aspect, this disclosure describes a method for detecting hepatocyte growth factor receptor (MET) in a sample. Generally, the method includes contacting a protein scaffold as summarized above with a sample that includes MET, allowing the protein scaffold to bind to MET in the sample, removing protein scaffolds that are not bound to MET, and detecting at least one protein scaffold:target molecule complex.

In another aspect, this disclosure describes a method that generally includes administering to a subject a pharmaceutical composition that includes a protein scaffold as summarized above in an amount effective to treat a condition treatable with the pharmaceutical composition.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Diversity gradients in binding molecules. (A) The Shannon entropy of antibody sequences (from the abysis database at http://www.bioinforg.uk/abysis/) in the framework (open circles, white background) and CDRs (solid circles, gray background). (B) The Shannon entropy of combinatorial library designs for antibody (data from A), fibronectin (Fn), designed ankyrin repeat (DARPin; Tamaskovic et al., 2012, Methods Enzymol. 503:101-134), affibody (Grimm et al., 2011, Mol Biotechnol. 48(3):263-276), and anticalin (Gebauer et al., 2012, Methods Enzymol. 503:157-188), domains. (C) The relative impact of alanine mutation on binding is shown for several protein interfaces: TEM1-β-lactamase (TEM1) and β-lactamase inhibitor protein (BLIP) (PDB: 1JTG); extracellular RNase (barnase) and its intracellular inhibitory binding partner (barstar) (PDB: 1BRS); light and heavy chain variable regions of anti-hen egg white lysozyme antibody D1.3 in the context of binding anti-D1.3 antibody E5.2 (not shown) (PDB: 1DVF); human growth hormone (hGH) and extracellular domain binding partner (hGHpb) (PDB: 1A22); heregulinβ (HRG) egf domain in the context of binding ErbB3 receptor-IgG fusion (not shown) (PDB: 1HAE).

FIG. 2. (A) The amino acid frequencies at the indicated sites for the first generation library (white) and the binding populations (black). Bars and error bars are mean±standard deviation. Statistical significance, while adjusting for family wise error rate using Bonferroni method, denoted at level α=0.005 (·). (B) Solution structure (PDB: 1TTG) of wild-type fibronectin domain with backbone residues of diversified loop sites denoted by spheres. BC, DE, and FG loops are labeled as are several sites for reference.

FIG. 3. The difference between the amino acid frequencies in the binding populations and the first generation library are shown for each amino acid at each site. The average across all fully diversified sites is also presented.

FIG. 4. Loop length frequencies in naive and binder populations as identified by DNA sequencing. Sanger sequencing of first generation populations (57 naïve, 167 binder) and Illumina sequencing of second generation (details in Table 3). Bars and error bars represent mean±standard deviation.

FIG. 5. The amino acid frequencies at the indicated sites for the second generation library (white) and the binding populations (black). Bars and error bars are mean±standard deviation.

FIG. 6. The difference between the amino acid frequencies in the binding population and the second generation library are shown for each amino acid at each site. The average across all fully diversified sites is also presented.

FIG. 7. Cysteine frequency analysis. (A, B) Change in pairwise frequency of clones containing exactly two cysteines (high-affinity populations minus naive library) for the first generation (A) and second generation (B) libraries. (C) Frequencies of clones containing the indicated number of cysteines in initial libraries and binder populations.

FIG. 8. Clonal characterizations. (A) Thermal stabilities of wild-type (WT) fibronectin and Fn3HP are shown. Median (black line), second and third quartiles (gray box), upper and lower inner fences (vertical lines), and outliers (diamonds) are shown for a sampling of clones from binarily diversified traditional libraries (n=15, Trad. Bind.) and three populations of the current study: first generation library binders, second generation naïve library, and second generation library binders. (B) Affinity titrations. Yeast displaying Fn3HP variants were incubated with the indicated concentration of biotinylated target molecule. Binding was detected by streptavidin-fluorophore and flow cytometry. Data points are from a single representative experiment. Affinities were calculated as 150±60 pM (rabbit IgG, clone 0.6.2, black triangles), 4±3 nM (lysozyme, clone 0.6.3, gray squares), and 11 nM (MET, clone 3.4.3, white circles).

FIG. 9. (A) The a carbons of evaluated sites are shown in spheres colored based on Shannon entropy of binding sequences from the second generation campaigns. (B, C) The Shannon entropies at each site of second generation binders are plotted versus the number of amino acids that are tolerated at that site based on computational stability predictions. (B) Sites with solvent accessible surface area (in other fibronectin domain mutants)≧40%. Pearson coefficient=0.63. Slope=0.13. (C) Sites with solvent accessible surface area <40%. Pearson coefficient=0.22. Slope=0.04.

FIG. 10. Measuring the influence of library design metrics. Sitewise evaluation of theoretical stability upon mutation and natural sequence frequency, as well as overall amino acid prevalence at binding interfaces of antibodies (i.e. complementarity), generate sitewise amino acid frequencies. The ability of these frequencies—scaled linearly based on solvent exposure and target exposure (Equation 1)—to collectively mimic the observed sitewise amino acid distributions in binding populations is evaluated. (A) The optimal weights for each contributing data set as a function of exposure are shown. (B-D) For the indicated weights of each metric, the other free parameters were varied to optimize the match between modeled sitewise amino acid distributions and experimentally observed sequences. The qualities of the fits are presented as the number of standard deviations above the fit obtained if unbiased data are used (i.e. uniformly 5% amino acid diversity rather than stability, homology, and complementarity bias). (B) Relative success when limited to two data inputs. Exposure independent (α) and dependent (β) weights are varied, subject to the indicated average weight, to maximize fit. (C) Sensitivity of exposure independent weights (α). All α values are fixed as indicated (note that all α's sum to 1 so complementarity weight is implicit). Exposure dependent weights are varied to maximize fit. 55% complementarity, 45% natural sequence frequency, and 0% theoretical stability optimize fit. (D) As in (C) but with set β values and varied a values.

### DETAILED DESCRIPTION OF ILLUSTRATIVE EMBODIMENTS

This disclosure describes evolving a hydrophilic type III fibronectin domain that specifically binds MET (also known as hepatocyte growth factor receptor). The molecules can have use in a clinical and/or research and discovery setting.

Molecules that recognize certain targets specifically and with high affinity are useful for many clinical (e.g., diagnostic and/or therapeutic) and biotechnology applications. Typically, antibodies have been used for many of these applications, but antibodies have certain properties that may be drawbacks in certain applications. The limitations of antibodies have encouraged investigation toward alternative protein scaffolds that allow one to efficiently generate improved binding molecules. In the context of targeting solid tumors, for example, antibodies—which are typically about 150 kDa for immunoglobulin G (IgG)—can exhibit, due at least in part to their size, poor extravasation from vasculature, poor penetration through tissue, and/or long plasma clearance halftime, which can lead to poor signal-to-noise ratio, especially for diagnostic imaging. Antibodies also can exhibit thermal instability, which can lead to a loss of efficacy as a result of denaturation and/or aggregation. In addition, antibodies are typically made in mammalian cultures because many possess disulfide bonds, glycosylation, and/or multi-domain structures. This intricate structure can interfere with engineering the antibody for a particular application such as, for example, production of protein fusions for bispecific formats. Moreover, the presence of disulfide bonds in antibody molecules often precludes their intracellular use.

As a result of the limitations inherent to antibodies, alternative protein scaffolds have been developed in attempts to address many or all of these shortcomings. This disclosure describes recombinant, non-naturally occurring fibronectin scaffolds capable of binding a compound of interest. In particular, the fibronectin scaffolds described herein may be used to display defined loops that are analogous to the complementarity-determining regions (“CDRs”) of an antibody variable region. These loops may be subjected to randomization or restricted evolution to generate the diversity required to build a library of fibronectin scaffolds that, while each scaffold binds to a specific target, the library, collectively, binds to a multitude of target compounds. The fibronectin scaffolds may be assembled into a multispecific scaffold capable of binding different two or more targets. The fibronectin scaffolds described herein can therefore provide functional properties typically associated with antibody molecules. In particular, despite the fact that the fibronectin scaffold is not an immunoglobulin, its overall folding is similar in relevant respect to that of the variable region of the IgG heavy chain, making it possible for a protein scaffold to display loops in relative orientations analogous to antibody CDRs. Because of this structure, the fibronectin scaffolds described herein possess ligand binding properties that are similar in nature and affinity to the binding properties of antigen and antibody. Also, loop randomization and shuffling strategies may be employed in vitro that are similar to the process of affinity maturation of antibodies in vivo.

The engineered fibronectin scaffolds described herein can provide a platform upon which amino acid diversity can be introduced to develop novel function. In some embodiments, a fibronectin scaffold can be efficiently evolvable to bind with the affinity and specificity typical of antibodies, but be more stable and/or exhibit better biodistribution than that typically exhibited by antibodies. As a result, the fibronectin scaffold may be useful in a wider range of applications and settings than a corresponding antibody molecule. In some embodiments, a fibronectin scaffold can be efficiently evolved to bind specifically to a target with a desired affinity, which in many applications may be characterized by a nanomolar to picomolar dissociation constant. High affinity and specificity can provide targeted delivery and/or reduce side effects in clinical applications. A fibronectin scaffold free of disulfide bonds allows for bacterial production in the reducing E. coli cellular environment, intracellular stability in mammals, and/or ease of chemical conjugation. A fibronectin scaffold benefits from retaining the native structure of its structural (a helix and/or β structure) and stability through the numerous possible mutations to the variable loop domains that can confer binding specificity.

The fibronectin scaffolds described herein can serve as a platform for ligand discovery towards a broad range of clinical, scientific, and/or industrial targets. For example, fibronectin scaffolds can possess permeability and/or distribution properties that make them suitable for, for example, targeting nascent tumors. Scaffolds also can be suitable for targeting atherosclerotic plaque and other biologically distinct vascular states. In other embodiments, fibronectin scaffolds can be suitable for drug delivery to the central nervous system for treatment and/or diagnosis of neurological disorders or diagnosis of neurobiological status. Fibronectin scaffolds also can be suitable for delivery to immune cells for immune modulation and/or immune surveillance. As yet another example, fibronectin scaffolds can be suitable for delivery to stem cells for modulation and/or diagnosis of cellular status.

This disclosure describes a platform for engineering a small (approximately 5-10 kDa), stable fibronectin scaffolds capable of efficient modification to generate target-specific picomolar affinity that can provide for sustained delivery in vivo. As used herein, “specific” and variations thereof refer to having a differential or a non-general affinity, to any degree, for a particular target. Generally, the fibronectin scaffolds described herein may be used to display defined loops that are analogous to the complementarity-determining regions (“CDRs”) of an antibody variable region. The variability of the loop regions permit generating fibronectin scaffold molecules that can specifically bind to any target of interest. The loops maybe subjected to randomization or restricted evolution to generate sufficient diversity that a library of fibronectin scaffold molecules can include sufficient members that the library, as a whole, can bind to a multitude of targets. Moreover, the fibronectin scaffolds may be assembled into a multispecific scaffold—e.g., a multimeric scaffold—capable of specifically binding two or more different targets.

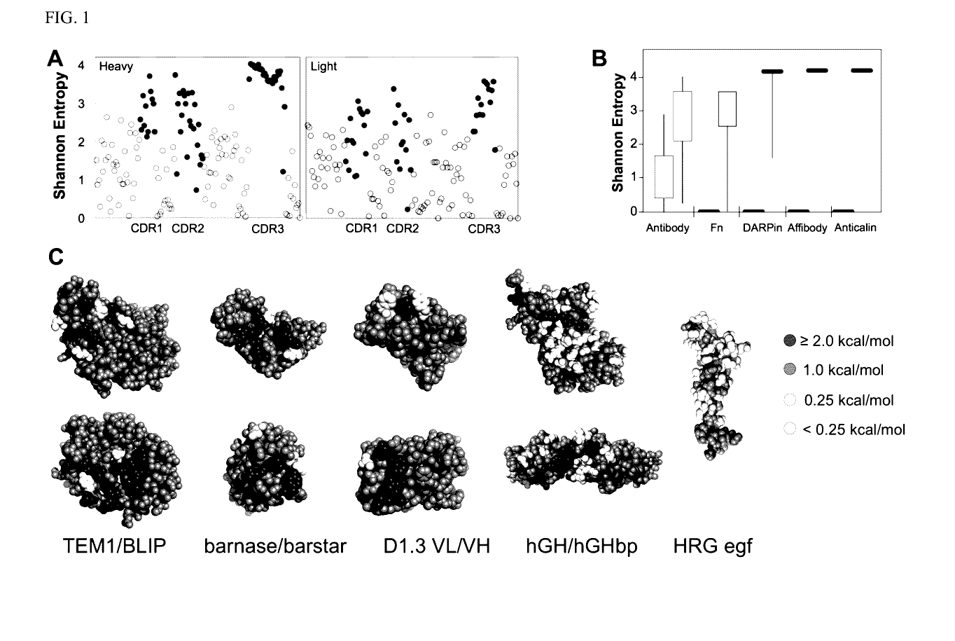

Combinatorial protein libraries can benefit from sitewise optimization across a gradient of amino acid diversity—akin to natural binder libraries (naïve antibody repertoires, FIG. 1A)—rather than the spatially binary design of a heavily diversified paratope and fully conserved framework predominant in synthetic scaffolds (FIG. 1B). Evolutionary efficiency may be optimal with complementarity-biased amino acid diversity in the binding hot spot, conserved wild-type sequence in the distal framework, and/or a gradient at intermediate sites including bias for conservation or interactive neutrality in proximal regions. This gradient is not purely spatial as protein structure and protein-protein interfaces are complex (FIG. 1C). Moreover, for novel ligand discovery, the exact paratope is not known ahead of time, which makes designed localization of a hot spot difficult.

Sitewise library optimization was studied across a gradient of diversity levels in the context of the type III fibronectin domain, a 10 kDa beta sandwich, diversified in three solvent-exposed loops termed BC, DE, and FG for the β-strands they connect. Diversification of one, two, or three loops, or the sheet surface, allows evolution of binding to a host of molecular targets. Diversification of two loops is evolutionarily superior to one-loop mutation, and although diversification of the third loop (DE) is not requisite for high-affinity binding, it can aid stability. Further stability bias at select positions—identified by natural sequence frequency, experimental stability analysis, and solvent exposure—was effective in library design. Moreover, antibody-inspired amino acid bias in putative hot spots can be effective within fibronectin libraries. The fibronectin domain was evolved for hydrophilicity to improve processing and in vivo biodistribution. The current study aims to expand upon these library developments for technological benefit and elucidation of evolutionary design principles as well as to provide analytical techniques for library design and evaluation. The extent of diversification and the extent of sitewise amino acid distributions that optimize evolutionary efficiency were studied in the hydrophilic fibronectin mutant (Fn3HP).

One can use high-throughput discovery and directed evolution of numerous binding ligands to various targets from a diverse combinatorial library followed by thorough sequencing of the library and binder populations to identify diversities and amino acids consistent with functional fibronectin domains. Deep sequencing of evolved protein populations has proven effective for analysis of functionality landscapes for maturation of single protein clones, protein families, and antibody repertoires. A modified approach was applied to identify optimal diversification strategies for synthetic naive combinatorial library design. The results demonstrate a range of diversities and sitewise amino acid preferences. Moreover, the optimized library provides stable, high affinity binders directly without maturation, and the sequence analysis provides a metric to evaluate the balance of inter- and intra-molecular considerations in library design, which are quantitatively assessed.

### Library Design and Construction

A combinatorial library was created with various levels of diversity throughout the potential paratope of the solvent-exposed loops of fibronectin (Table 1). Each loop was also allowed to vary in length as guided by natural sequence frequency. The core of the BC and FG loops, 4-11 sites depending on loop length diversity, had full amino acid diversity biased to mimic the third heavy chain complementarity-determining region (CDR) of antibodies. One excepted site, V29, has been shown to benefit from constraint as a small, reasonably hydrophobic amino acid so constrained diversity (A, S, or T) was permitted. A second exception, G79, has been shown to benefit from glycine bias. To increase glycine frequency while mimicking CDRs, the site was mildly constrained to G, S, Y, D, N, or C. Twelve sites adjacent to the core of the BC and FG loops were afforded five levels of diversity: i) wild-type, ii) wild-type or serine (as a small, mid-hydrophilicity neutral interactor), iii) wild-type, serine, or tyrosine (the most generally effective side chain for complementarity), iv) moderate chemical diversity

(A, C, D, G, N, S, T, or Y), or v) full antibody-mimicking amino acid diversity. Five sub-libraries were constructed using separate DNA oligonucleotides with the appropriate degenerate codons for a single level of diversity. The five sub-libraries for each of the three loops were pooled in an equimolar fashion.

The gene libraries were transformed into a yeast surface display system (Boder et al., 1997, Nat Biotechnol. 15(6):553-557), which yielded 2.0×10⁸ transformants. DNA sequencing of 57 randomly selected naïve clones indicated 61% had full-length sequences, 16% contained stop codons naturally arising from the CDR' diversity, and 21% contained frameshifts. This finding was supported by flow cytometry analysis that revealed 64% of proteins were full-length as evaluated by the presence of a C-terminal c-myc epitope. Thus, the library contained 1.2×10⁸ unique, full-length Fn3HP clones.

Selection and Analysis of Binding Populations from First Generation Library

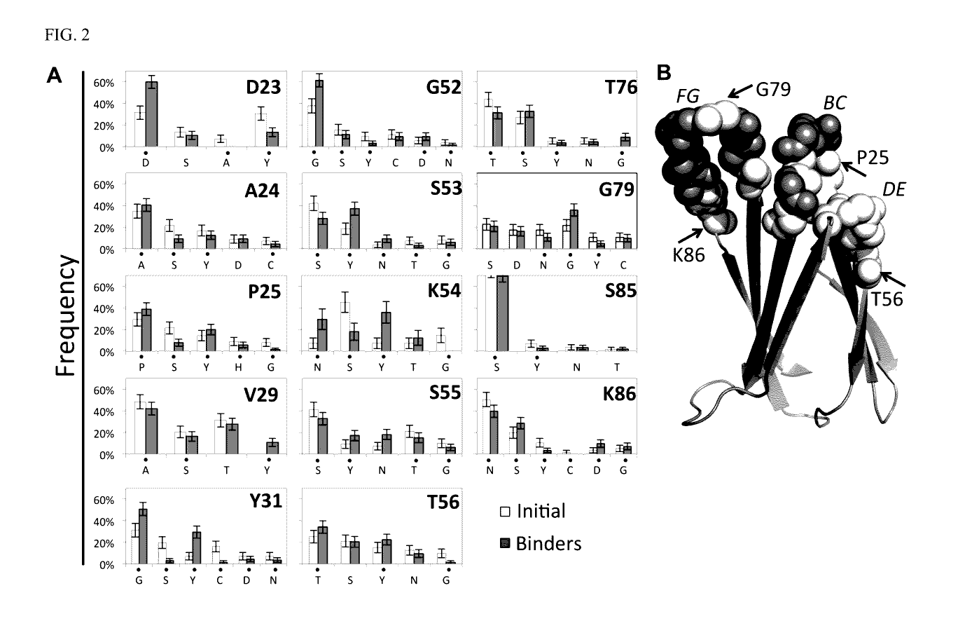

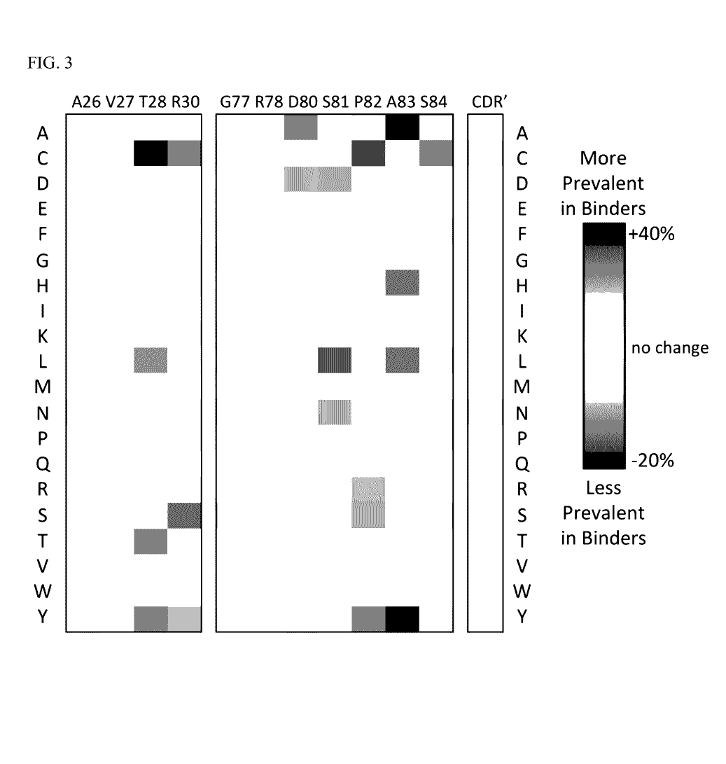

The pooled library was sorted, using magnetic beads with immobilized protein targets and fluorescence-activated cell sorting (FACS), and evolved to identify a diverse set of selective binders to goat immunoglobulin G (IgG), rabbit IgG, lysozyme, or transferrin. Following a single round of mutagenesis, then two rounds of magnetic bead sorting, an enriched population of mutants was isolated that demonstrated mid-affinity, selective binding to transferrin. This population was then sorted for high-affinity binders via FACS. Similarly, though with one additional round of mutagenesis, mid- and high-affinity, selective binders for goat IgG, rabbit IgG, and lysozyme were identified. An initial sampling of 167 clones was sequenced from binding populations. Binders of each target exhibited broad diversity across each of the three loops. Goat IgG, rabbit IgG, lysozyme, and transferrin binders demonstrated 74%, 61%, 42%, and 84%, uniqueness, respectively. Sitewise comparison of amino acid frequencies before (57 naïve clones sequenced) and after binder selection provides information on the ideal amino acid diversities at each site within Fn3HP (FIG. 2 and FIG. 3). Sitewise analysis was used to design a second generation library to provide further refinement of the diversity distributions.

Within the BC loop at site 23, wild-type D was enriched from 31±6% in the naïve library to 60±6% in binders. S was maintained (13±5% to 10±4%), whereas Y (30±6% to 13±4%) and A (7±3% to 0±0%) were depleted. 17±2% of mutants had amino acids from the more diverse sublibraries (vs. 18±2% naive). Natural fibronectin homolog sequence frequency data are in agreement, placing aspartic acid as the most prevalent residue at site 23. Thus, the second generation library fully conserved wild-type D at site 23.

At site 24, wild-type A was slightly elevated (35±6% to 40±6%). Y was weakly depleted (17±5% to 13±4%) whereas S was substantially depleted (21±5% to 9±3%). 29±3% of binding mutants had amino acids from the more diverse libraries (vs. 18±2% naive). Thus, the options of wild-type conservation and mild diversity were further explored in the second generation.

At site 25, wild-type P was enriched from 29±6% in the naive library to 39±6% in binders. While tyrosine was enriched from 14±5% to 20±5%, serine declined from 21±5% to 8±3%. 26±3% of mutants had amino acids from the more diverse libraries (vs. 28±3% naive).

Wild-type conservation appears beneficial at site 25 within the BC loop. Thus, the second generation compared two library designs: fully conserved P or PYSH diversity. As used herein, in the context of discussing diversity at a specified site, a series of unseparated amino acid abbreviations refers to equally possible amino acids at the given position. For example, PYSH indicates a 25% possibility of each of proline, tyrosine, serine, and histidine at that site.

At site 29, the initial library frequencies for small residues A, S, and T of 48±7, 20±5, and 31±6% were generally conserved in the binding population at 42±6, 16±4, and 28±5%, respectively. Tyrosine, achievable via mutation or erroneous synthesis, was observed among binders at 11±4%. While the increase in tyrosine is notable, including this residue in subsequent library designs may encounter potential detriment as codon synthesis would be constrained to include the charged and acidic residues D and N. Thus, library design at site 29 maintained a distribution of AST.

At site 31, wild-type Y was enriched from 7±3% in the naive library to 29±6% in binders. Furthermore, glycine, which occurs with 31% frequency at this site within natural sequences of homologous proteins, increased in prevalence from 31±6% to 51±6%. Alternatively, substantial decreases in both serine (20±5% naive; 3±2% binders) and cysteine (16±5% naive; 1±1% binders) were observed. The second generation library contained GY diversity.

At site 52, wild-type G was enriched from 38±7% in the naive library to 61±6% in binders. S was nearly maintained (16±5% to 11±4%) in binders whereas Y was depleted (9±4% to 3±2%). Only 5±1% of mutants had amino acids from the more diverse libraries (vs. 17±2% naïve). Wild-type conservation appears strongly beneficial at site 52.

At sites 53-55, Y and N were enriched whereas S was depleted, but still present at reasonable levels. Thus, the second generation library implemented YNST diversity at these sites.

At site 56, wild-type T (25±6% to 34±6%) and Y (15±5% to 23±5%) were elevated while N (13±4% to 10±4%) and S (21±5% to 20±5%) were maintained leading to TYSN design.

Within the FG loop at site 76, wild-type T was depleted from 44±7% in the naive library to 31±6% in binders. S was maintained at a high level (27±6% to 33±6%) whereas Y was maintained at a lower level (5±3% to 4±2%) in binders. Additionally, G increased by 9±3% (not represented within naive library sample of n =57). Thus, the next design included TSGA.

At site 79, wild-type G was enriched (22±5% to 36±6%), S (23±6% to 21±5%) and D (17±5% to 16±4%) were maintained, and Y (10±4% to 5±3%) and N (17±5% to 11±4%) decreased. Thus, GSDN diversity was used in the second library.

At site 85, wild-type S maintained (74±6% to 69±5%), which prompts future conservation. At site 86, N was mildly decreased (51±7% to 40±6%) while S increased from 20±5% to 29±5% and Y decreased from 10±4% to 3±2%. The second generation design was synthesized as conserved N.

In evaluating the fully diversified sites (FIG. 3), several elements are noteworthy. In addition to the aforementioned enrichment of glycine at sites 76 and 79, sites 77 and 78 also elevate glycine (3±2% to 13±4% and 7±3% to 11±4%). At G77, Y was present at 16±5% initially and 22±5% in binders and A, D, and T were each maintained (28±11% overall to 32±11%). Thus, G77 was set to GSYADTNC in generation two. Though several enrichments and depletions are evident elsewhere, all other sites will be maintained as CDR'. The antibody-inspired diversity was essentially maintained with slight reductions in C (9±1% to 5±0%) and N (9±1% to 3±0%) and enrichments in D (11±1% to 16±1%) and G (4±0% to 8±0%).

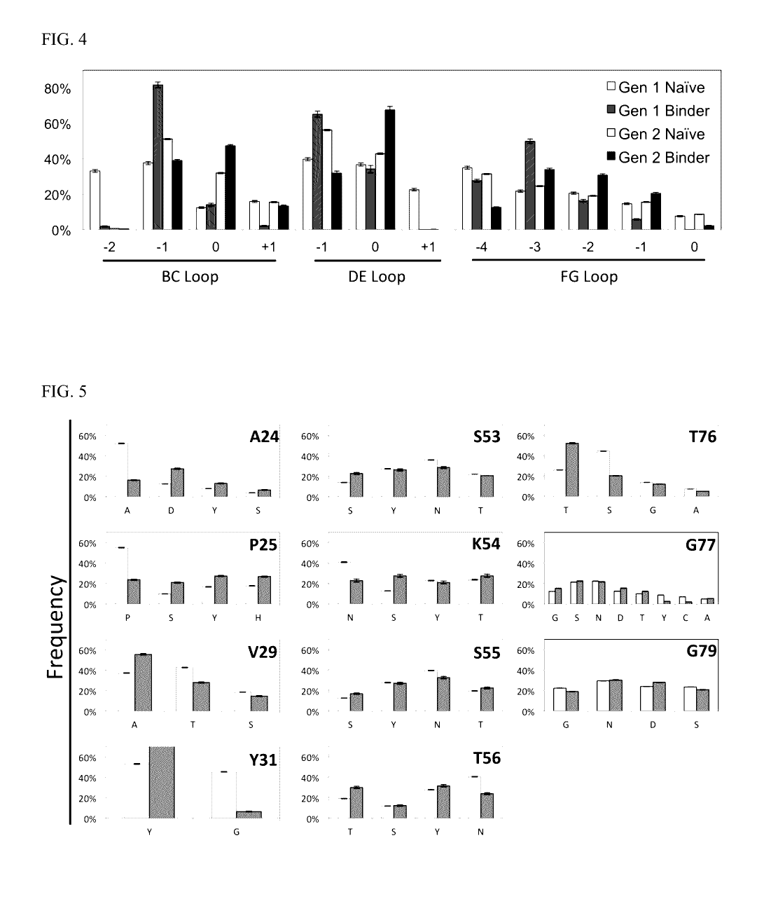

While most of the allowed loop lengths were observed in the binding populations (FIG. 4) the shortest BC loop (two amino acid deletions from wild-type) and extended DE loop were rarely observed and, thus, omitted from future consideration.

Construction, Selection, and Analysis of Binding Populations from Second Generation Library

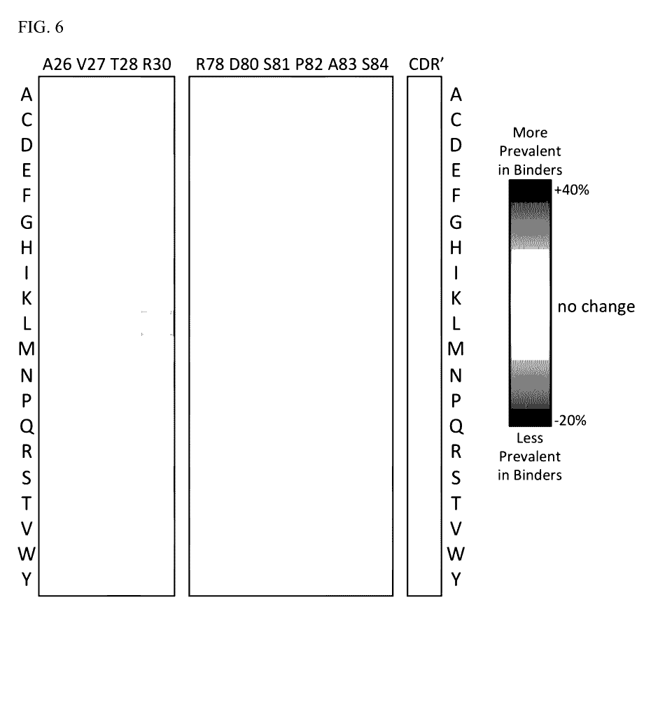

The second generation library (Table 2) was constructed from degenerate oligonucleotides. 4.2×10⁹ yeast transformants were obtained. 71% were full-length as assessed by cytometry and corroborated by Sanger sequencing where 67% of clones were full-length. Mid- and high-affinity binders to MET, lysozyme, and rabbit IgG, as well as mid-affinity binders for tumor necrosis factor receptor superfamily member 10b, were evolved and sequenced using Illumina MiSeq with barcodes to identify mid- and high-affinity binders. Sequences were aligned, clustered, and counted, with accommodations to reduce overcounting of highly similar sequences. 484,000 sequences were collected with 232,000 identified as unique (Table 3). The sitewise differences between amino acid frequencies in the naive library and selected binders were calculated at constrained sites (FIG. 5) and CDR′ sites (FIG. 6).

Sites 24 and 25 exhibit similar results in which significant wild-type conservation was not maintained in binders (52% to 16% of A24 and 55% to 24% of P25) and the other amino acid options were elevated fairly uniformly. At site 29, alanine was increased (37% to 56%) while threonine was depleted (43% to 28%). At site 31, wild-type tyrosine is substantially enriched (53% to 92%) at the expense of glycine (45% to 7%).

In the middle of the DE loop, at sites 53-55, asparagines were depleted from their overly high starting points (36% to 29%, 41% to 23%, and 40% to 33%) while serines, which were more rare than designed in the naive library, were increased (14 to 23%, 13% to 27%, and 13 to 17%). Y and T were essentially maintained thereby supporting the SYNT diversity when equally implemented. Asparagine was also decreased at site 56 (41% to 24%), but wild-type threonine was preferentially increased (19% to 30%).

At the edge of the FG loop, wild-type T76 was increased from 26% to 52% in binders while serine was decreased from 44% to 20%. At site 77, wild-type G (12% to 15%) and aspartic acid (12% to 16%) were enriched, serine (21% to 22%) and asparagine (22% to 22%) were maintained, and tyrosine (9% to 3%) and cysteine (7% to 2%) were depleted. At site 79, the GDSN diversity was consistently maintained in binders.

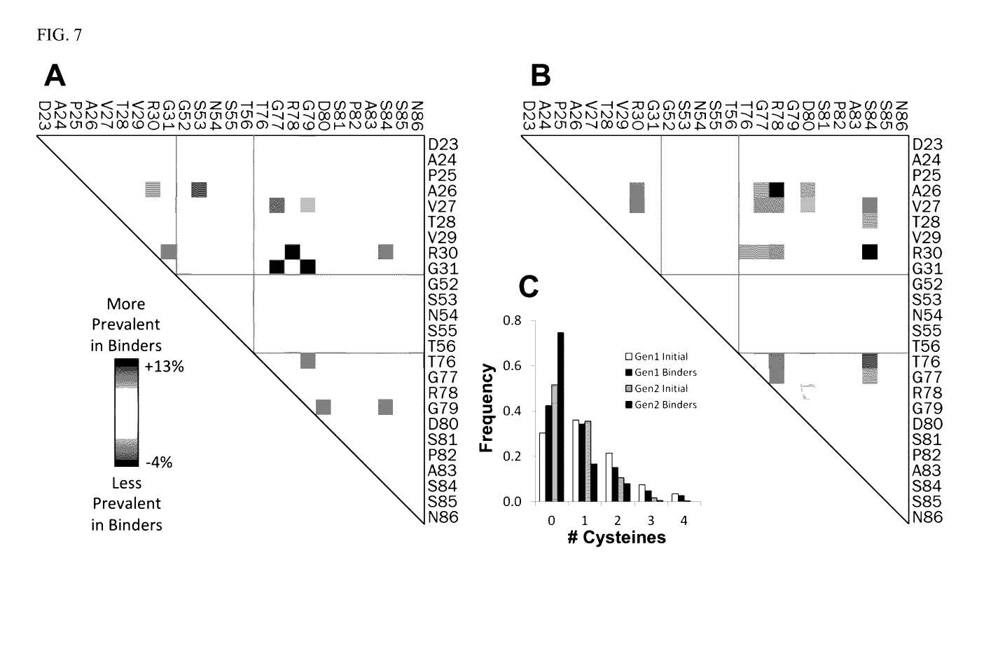

In the fully diversified sites, the antibody-inspired diversity was maintained for many amino acids. Sitewise exceptions include wild-type conservation at D80 (9% to 22%), T28 (4% to 11%), and S81 (14% to 18%) and enrichment of isoleucine and leucine at site 30 (4% to 38%). Slight overall exceptions—e.g., decrease in cysteine (7% to 4%) and increases in hydrophobics isoleucine, leucine, and valine (sum 8% to 15%)—compensate for imperfections in the degenerate codons, yielding frequencies more in line with natural antibody repertoires. The decrease of cysteine residues is driven by a lack of enrichment of single-cysteine clones more than depletion of dual-cysteine clones, which is perhaps suggestive of beneficial disulfide bond formation (FIG. 7). Evaluation of cysteine pairs in dual-cysteine clones indicates a strong enrichment of clones with a cysteine in sites 26-28, especially 27, of the BC loop and 80-84 of the FG loop. Simultaneous cysteines at sites 76 and 84 are also frequently selected in binders.

Loop length analysis indicated diverse lengths were used in binders with a preference for wild-type lengths in the BC and DE loops and a removal of two or three amino acids in the FG loop (FIG. 4). The longest FG loop, which is only observed in the tenth type III domain of human fibronectin but not the other fourteen human type III domains, is rarely observed in binders. The shortest FG loop was also less frequently observed in binders than in the unsorted library.

The framework sites that were intended for conservation were also analyzed within the naive and binder sequences to identify mutations, occurring during oligonucleotide synthesis, gene assembly, or directed evolution, that were preferentially present in binding clones. Five mutations were enriched (Table 4).

### Binder Phenotypic Characterization

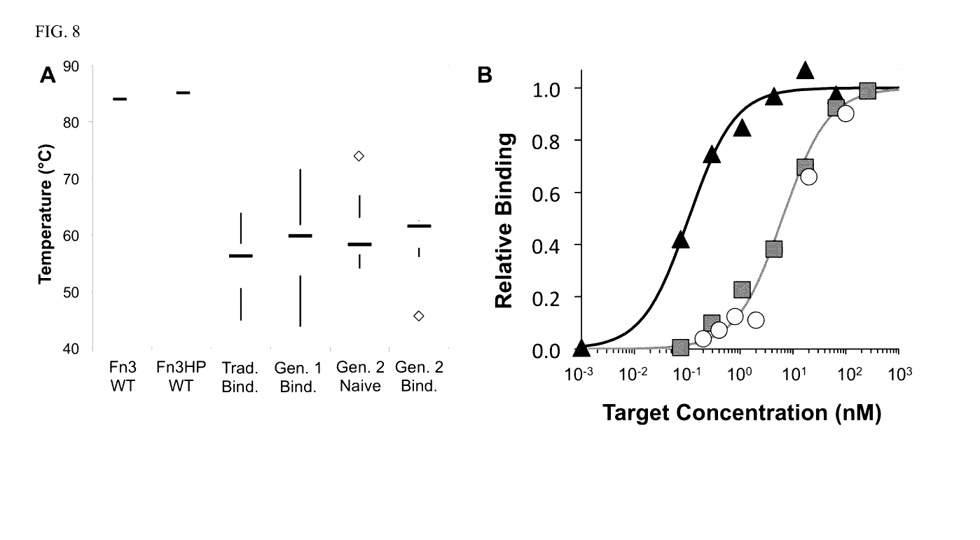

By constraining diversity at select sites, one can improve the balance of inter- and intra-molecular interaction evolution and reduce destabilization upon mutation. Thus, the stability of several fibronectin mutant populations were evaluated: binders from both library generations in this work and binders evolved from previous binary (fully conserved framework, fully diversified loops) libraries from previous literature as well as the naive second generation population and the parental fibronectin domains (human and hydrophilic mutant) (FIG. 8A). The first, second, and third quartile stabilities are higher for the first and second generation libraries relative to binders from less biased libraries. Further still, the median stability of the less biased library binders is less than even that of the naive members of the second generation library (p<0.05). Fn3HP is of essentially equivalent stability as Fn3 (84° C. vs. 85° C.).

In addition to yielding stable binders, the second generation library yields high-affinity binders with little to no evolution. Three binder campaigns continued with additional sorts to identify the strongest binders in the population. Rabbit IgG and lysozyme binders were characterized following two rounds of magnetic bead selection, one round of cytometry sorting at target concentrations of 50 nM and a final round of cytometry sorting at 1 nM, wherein the top 1% of binding events were isolated. Titrations curves of representative clones from the most stringently sorted, non-evolved rabbit IgG and lysozyme populations (FIG. 8B) revealed affinities of 150±60 pM and 4±3 nM, respectively. High-affinity MET binders were isolated following three iterations of evolution, each iteration consisting of two magnetic bead selections, one cytometry sort for full-length clones, and one round of error-prone PCR. A representative clone from the evolved MET binding population yielded an affinity of 11 nM (FIG. 8B).

### Binder Characteristics Beyond Single Amino Acid/Single Site Preferences

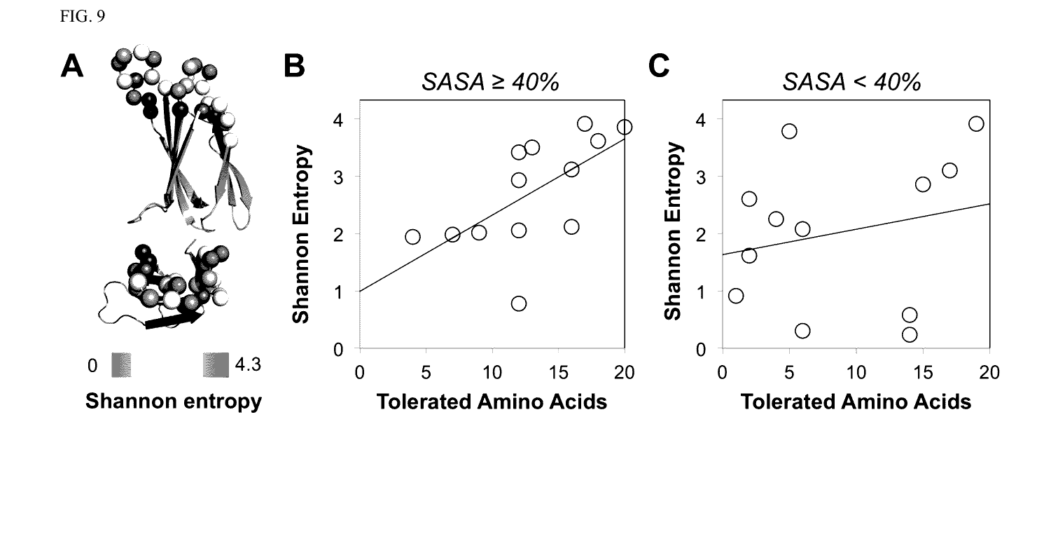

The binders generated from the second generation library exhibit a range of sitewise diversity that is not purely spatial (FIG. 9A). Four sites exhibit Shannon entropies in excess of 3.5. Three additional sites are in excess of 3.0. Nine sites have entropies from 2.0-3.0. Six diversified sites exhibit Shannon entropies below 2.0. Four sites were conserved, as designed, based on first generation library analysis.

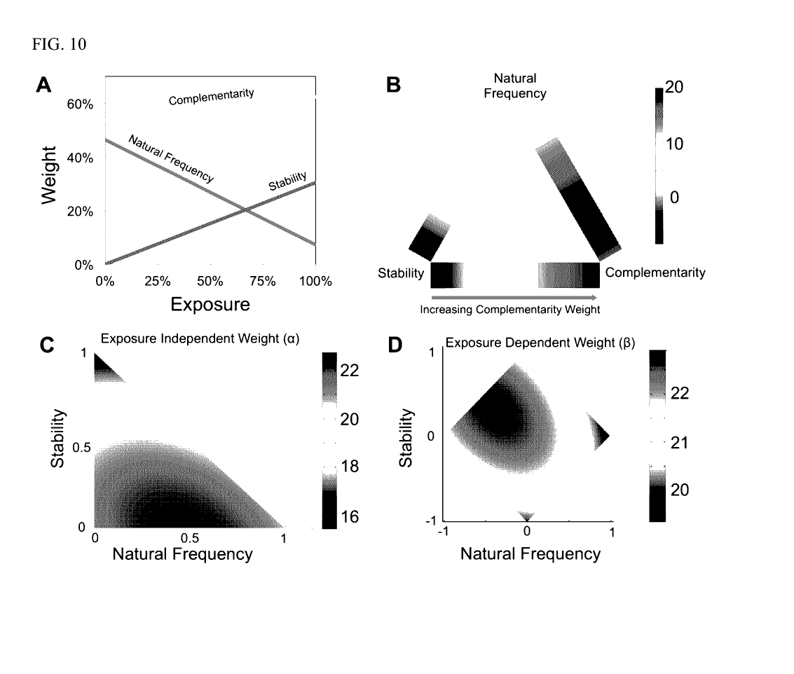

The high-throughput binder engineering and analysis described herein provides one way of identifying the extents of diversification, as well as the relevant amino acids, at each site. One can examine whether any computational means could have guided this library refinement. The Shannon entropies of evolved binders at exposed sites (FIG. 9B) correlated with theoretical mutational tolerance whereas no correlation was observed for less exposed sites (FIG. 9C). More broadly, four elements were considered for their ability to drive effective library design: sitewise amino acid frequencies from natural fibronectin homologs, theoretical stability of each amino acid at each site within the context of diverse fibronectin clones, complementarity-biased amino acid distributions observed in antibody CDRs, and residue exposure. The ability of these first three elements to combine to generate sitewise amino acid distributions that matched the experimentally observed frequencies was examined. The relative weights of the first three elements were allowed to vary for each site based purely on the solvent and target accessibility of that site. Target accessibility was scored based on the orientation of the amino acid side chain relative to the rest of the diversified paratope. Weighted inclusion of all elements allows library designs that perform 22 standard deviations above unbiased input matrices (i.e., input data contains uniformly 5% amino acid distribution) (FIG. 10). For well-exposed sites, amino acid diversity is effectively mimicked by 62% complementarity bias, 30% stability computation, and 8% natural frequency. At sites with less exposure, natural amino acid frequency should be more heavily weighted at the expense of stability and, less so, complementarity (FIG. 10A). Design based on a single element is inferior to randomness for stability, marginally effective (2 standard deviations above random) for natural frequency, and reasonably effective (16 standard deviations above random), but significantly suboptimal, for complementarity (FIG. 10B).

In the pursuit of a broadly functional combinatorial library capable of yielding binders to numerous targets, the benefit of diversification is unclear for sites peripheral to a ‘hot spot’ that enthalpically drives high-affinity binding. These peripheral sites can (a) directly contact target, (b) impact neighboring residue orientation to improve interfacial enthalpy upon binding, (c) impact neighboring residue orientation to reduce entropic penalty upon binding, and/or (d) stabilize the protein. Yet these potential benefits can be offset by the inverse impacts: make unfavorable interfacial contact, worsen neighboring residue orientation, and/or destabilize the protein. If sufficient ‘hot spot’ interfacial area is not yet present for high-affinity binding, then additional sites must be diversified to enable favorable interaction. At some point, this expanded paratope provides sufficient interface for strong, specific affinity. Similar tradeoffs can be considered for peripheral sites. Given the typical detriment of random mutation, the average peripheral mutation will hinder all four elements thereby suggesting against diversity. Though as a corollary, on average, mutations in the ‘hot spot’ will negatively impact the last two elements by worsening the entropic penalty upon binding and destabilizing the protein because of imperfect interactions with the conserved peripherals. Thus, peripherals need to be chosen to make neutral to good contact with: the intermolecular target (in the context of (a), above); intramolecular neighbors involved in binding (In the context of (b) and (c), above); and/or all intramolecular neighbors (in the context of (d), above). For considerations (a)-(c), above, since beneficial interactions will be unlikely, amino acids—e.g., serine—that yield relatively neutral interactions may be preferred. For consideration (d), beneficial interactions are likely for the wild-type residue and conserved neighbors based on their coevolution so conservation should be the aim. Since precise locations of neither the hot spots nor these transitions are known for each new ligand-target interface, the most efficient evolution may be achieved with a combinatorial library exhibiting a gradient of diversity from extensive diversity in the potential paratope hot spot to full conservation in the framework. Importantly, this gradient includes moderate diversity, with structural bias, within the paratope interfacing with target yet peripheral to the hot spot. Moreover, more mild diversity is included adjacent to the interfacial residues to yield optimal intramolecular contacts with the newly identified paratope. The range of Shannon entropies (FIG. 9) and amino acid frequency distributions (FIG. 2, FIG. 3, FIG. 5, and FIG. 6) across many binding sequences against several targets support the benefit of a gradient of diversities within combinatorial libraries. The particular amino acid distributions ae consistent with benefits resulting from wild-type conservation, serine bias, and/or complementarity bias, at appropriate sites.

Sitewise optimization of this gradient between intra- and inter-molecular interaction bias can be achieved with high-throughput binder generation and sequencing as demonstrated here. Yet this requires a sufficiently effective library to generate numerous binders, which may be difficult for new scaffolds or paratopes. Initial combinatorial library design can be guided by complementarity-determining residues and, when available, natural sequence frequencies, stability data (theoretical or experimental), and side chain exposure to solvent and target (FIG. 10).

Sitewise optimization of amino acid frequency, with a range of diversities, can be implemented in numerous ways. Trimer phosphoramidite codons can be used in oligonucleotide synthesis, which enables precise control over each distribution but elevates synthesis complexity and cost. Independent oligonucleotides can be synthesized for each loop sequence, which further elevates control by enabling pairwise (and higher order) site design albeit at an elevated synthesis scope. Simpler, less expensive single-nucleotide mixed degenerate oligonucleotide synthesis can approximate many amino acid distributions, especially with the inclusion of unbalanced nucleotide frequencies as used in this study. The amino acid distribution within antibody CDR-H3 can be closely approximated by unbalanced single-nucleotide methods, ¹⁴ but it must compromise on the genetic code connectivity of glycine, tyrosine, and cysteine. Achieving the desired high frequencies of tyrosine (20%) and glycine (16%) yields much more cysteine than desired (10%). In these libraries, high tyrosine (17%) frequencies were maintained while limiting cysteine (5%) at the expense of low glycine (4%). In the first generation library, which only had moderate glycine bias at G52 and G79, glycine was increased in binding sequences relative to the initial library (8±0% vs. 4±0% in fully diversified sites). Yet, with increased wild-type bias at G52 (100% G), G77 (17% G), and G79 (25% G) in the second generation, the glycine frequency in fully diversified sites within binders decreased to 3%. Thus, the presence of glycine within the loops, particularly DE and FG, clearly benefits binding evolution of the fibronectin domain; this glycine presence can be effectively achieved with sitewise bias.

The sublibrary synthesis approach in generation one (Table 1) yields coupling between sites within each loop. For example, wild-type D23 conservation pulls wild-type conservation in other BC sites during generation one analysis. In the absence of this coupling in generation two analysis, wild-type conservation at other BC sites (A24, P25, and Y31) is reduced. In the DE loop, wild-type G52 conservation pulls N54 conservation, which converts to N54 depletion in generation two in the absence of G52 coupling. Thus, when evaluating a new scaffold or paratope design, sublibrary construction enables analysis of numerous diversification strategies, but care must be taken to consider coupled sites.

While cysteines were overall depleted from binding sequences relative to the naive library, select inter- and intra-loop cysteine pairs were enriched. These occurred at proximal locations that are structurally sensible for disulfide bond formation. Enhanced evolutionary efficiency of this class of clones warrants consideration of biased design to drive the conformational restriction beneficial to numerous topologies including stapled helical peptides, shark new antigen receptors, camelid antibody domains, and previous fibronectin clones. Yet, while entropically beneficial, this conformational restriction may limit the diversity of paratopes that a library can present. Moreover, it eliminates the benefits of cysteine-free ligands:

intracellular use, efficient cytoplasmic production in bacteria, and genetically introduced cysteines for site-specific thiol chemistry.

This disclosure therefore describes recombinant, non-naturally occurring polypeptide scaffolds that specifically bind to hepatocyte growth factor receptor (MET). As used herein, “Specific,” “specifically,” and variations thereof refer to having a differential or a non-general affinity, to any degree, for a particular target. Generally, the scaffolds include a frame region and at least one loop region that specifically binds to MET. The loop regions can possess an amino acid sequence from, or derived from, a naturally occurring amino acid sequence. As used herein, an amino acid sequence “derived from” a naturally occurring amino acid sequence may exhibit one or more amino acid additions, amino acid substitutions, amino acid deletions, and/or post-translational modifications (collectively, “modifications”) to confer a desired functionality such as, for example, binding specificity and/or controlled reactability. Thus, one or more of the loop region amino acid sequences vary by deletion, substitution, and/or addition by at least one amino acid from the corresponding loop amino acid sequences of the naturally occurring protein from which it is derived. Similarly, the frame region of the protein scaffold can possess an amino acid sequence from, or be derived from, a naturally occurring amino acid sequence. In some embodiments, the frame region can possess, or be derived from, an amino acid sequence native to fibronectin.

In another aspect, this disclosure provides pharmaceutical compositions that include one or a combination of protein scaffolds described herein, formulated together with a pharmaceutically acceptable carrier. Such compositions may include one or a combination of, for example, two or more different protein scaffolds. For example, a pharmaceutical composition of the invention may include a combination of scaffolds that bind to different epitopes of hepatocyte growth factor receptor or that have complementary activities.

A pharmaceutical composition can be administered in combination therapy—i.e., combined with other agents. For example, a combination therapy can include a protein scaffold as described herein combined with at least one other therapy wherein the therapy may be immunotherapy, chemotherapy, radiation treatment, or drug therapy.

A pharmaceutical composition may include one or more pharmaceutically acceptable salts. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition also, or alternatively, may include a pharmaceutically acceptable anti-oxidant. Examples of pharmaceutically acceptable antioxidants include water soluble antioxidants such as, for example, ascorbic acid, cysteine hydrochloride, sodium bisulfate, sodium metabisulfite, sodium sulfite, and the like; oil-soluble antioxidants such as, for example, ascorbyl palmitate, butylated hydroxyanisole (BHA), butylated hydroxytoluene (BHT), lecithin, propyl gallate, alpha-tocopherol, and the like; and/or metal chelating agents such as, for example, citric acid, ethylenediamine tetraacetic acid (EDTA), sorbitol, tartaric acid, phosphoric acid, and the like.

A pharmaceutical composition also, or alternatively, may include an aqueous or non-aqueous carrier. Examples of suitable aqueous and non-aqueous carriers that may be employed in a pharmaceutical compositions include, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A pharmaceutical composition also, or alternatively, may include one or more adjuvants such as, for example, a preservative, a wetting agent, an emulsifying agent, and/or a dispersing agent. In some embodiments, a pharmaceutical composition can include an antibacterial agent and/or an antifungal agent such as, for example, paraben, chlorobutanol, phenol sorbic acid, and the like. It may also be desirable to include isotonic agents, such as sugars, sodium chloride, polyalcohols such as mannitol, sorbitol and the like into the compositions. In addition, prolonged absorption of an injectable pharmaceutical form may be provided by including an agent that delays absorption such as, for example, aluminum monostearate or gelatin.

A pharmaceutical composition typically is prepared to be sterile and stable under the conditions of manufacture and storage. A pharmaceutical composition can be formulated as a solution, a microemulsion, a liposome, or other ordered structure suitable to high drug concentration. A sterile injectable solution can be prepared by incorporating one or more protein scaffolds—including in some instances, one or more multimeric scaffolds—in an effective amount in an appropriate solvent with one or a combination of ingredients enumerated above, as desired, followed by sterilization microfiltration. Generally, a dispersion can be prepared by incorporating one or more protein scaffolds—including in some instances, one or more multimeric scaffolds—into a sterile vehicle that contains a basic dispersion medium and any other desired ingredients from those enumerated above. In the case of sterile powders for the preparation of sterile injectable solutions, vacuum drying and/or freeze-drying (lyophilization) can yield a powder of one or more protein scaffolds—including in some instances, one or more multimeric scaffolds—plus any additional desired ingredient from a previously sterile-filtered solution thereof.

To prepare pharmaceutical or sterile compositions including a protein scaffold, the protein scaffold can be mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms:

Parenteral Medications, Marcel Dekker, N.Y.; Lieberman, et al. (eds.) (1990) Pharmaceutical Dosage Forms: Tablets, Marcel Dekker, N.Y.; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Determining an appropriate dose can involve, for example, using parameters or factors known or suspected in the art to affect treatment or predicted to affect treatment. Generally, one can begin with an amount somewhat less than the anticipated optimum dose and thereafter increase the dose by small increments until the desired effect is achieved relative to any negative side effects.

Actual dosage levels of the active ingredients in a pharmaceutical composition as described herein may be varied so as to obtain an amount of the active ingredient that is effective to achieve the desired therapeutic response for a particular patient, composition, and mode of administration, without being toxic to the patient. The selected dosage level may depend, at least in part, upon a variety of pharmacokinetic factors including, for example, the activity of the particular composition being administered, the route of administration, the time of administration, the rate of clearance of the particular protein scaffold being employed, the duration of the treatment, other drugs, compounds and/or materials present in the pharmaceutical composition, the age, sex, weight, condition, general health and prior medical history of the patient being treated, and like factors well known in the medical arts.

An effective dose of a small molecule therapeutic such as a protein scaffold is typically about the same as for an antibody or polypeptide on a molar basis, but a lower dose may be effective on a mass basis. Moreover, still lower doses may be effective for diagnostic applications. Thus, a minimum effective dose can be at least 100 pg/kg body weight such, for example, at least 0.2 ng/kg, at least 0.5 ng/kg, at least 1.0 ng/kg, at least 10 ng/kg, at least 100 ng/kg, at least 0.2 μg/kg, at least 0.5 μg/kg, at least 1.0 μg/kg, at least 2.0 μg/kg, at least 10 μg/kg, at least 25 μg/kg, at least 100 μg/kg, at least 0.2 mg/kg, at least 0.5 mg/kg, at least 1.0 mg/kg, at least 2.0 mg/kg, at least 5.0 mg/kg, at least 10 mg/kg, at least 25 mg/kg, or at least 50 mg/kg (see, e.g., Yang, et al. 2003. New Engl. J. Med. 349:427-434; Herold, et al. 2002. New Engl. J. Med. 346:1692-1698; Liu, et al. 1999. J. Neurol. Neurosurg. Psych. 67:451-456; and Portielji, et al. 2003. Cancer Immunol. Immunother. 52:133-144). In some embodiments, the dosage may be, for example, from 0.1 μg/kg to 20 mg/kg, from 0.1 μg/kg to 10 mg/kg, from 0.1 μg/kg to 5 mg/kg, from 0.1 to 2 mg/kg, from 0.1 μg/kg to 1 mg/kg, from 0.1 μg/kg to 0.75 mg/kg, from 0.1 μg/kg to 0.5 mg/kg, from 0.1 μg/kg to 0.25 mg/kg, from 0.1 μg/kg to 0.15 mg/kg, from 0.1 μg/kg to 0.10 mg/kg, from 0.1 μg/kg to 0.5 mg/kg, from 0.01 mg/kg to 0.25 mg/kg, or from 0.01 mg/kg to 0.10 mg/kg of the patient's body weight.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the protein scaffold may be administered, for example, from a single dose to multiple doses per week, although in some embodiments the method can be performed by administering the protein scaffold at a frequency outside this range. In certain embodiments, the protein scaffold may be administered from about once per month to about five times per week.

A composition also may be administered via one or more routes of administration using one or more of a variety of methods known in the art. As will be appreciated by the skilled artisan, the route and/or mode of administration will vary depending upon the desired results. Exemplary routes of administration for scaffolds of the invention include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically.

If a pharmaceutical composition that includes one or more protein scaffolds described herein is administered in a controlled release or sustained release system, a pump may be used to achieve controlled or sustained release. Alternatively, polymeric materials can be used to achieve controlled or sustained release of a pharmaceutical composition that includes a protein scaffold. Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring less of the therapeutic protein scaffold composition in order to achieve the desired therapy.

If the protein scaffold described herein is administered topically, it can be formulated in the form of an ointment, cream, transdermal patch, lotion, gel, shampoo, spray, aerosol, solution, emulsion, or other form well-known to one of skill in the art. For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the protein scaffold described herein is administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

### Methods of Using Protein Scaffolds

In yet another aspect, this disclosure provides imaging methods and methods of treating, ameliorating, detecting, diagnosing, or monitoring a disease or a symptom or clinical sign thereof, as described herein, in a patient by administering therapeutically effective amounts of a protein scaffold described herein and/or a pharmaceutical composition that includes one or more protein scaffolds described herein.

As used herein, the term “treating” and variations thereof refer to reducing, limiting progression, ameliorating, or resolving, to any extent, the symptoms or clinical signs related to a condition. A “symptom” refers to any subjective evidence of disease or of a patient's condition; a “sign” or “clinical sign” refers to an objective physical finding relating to a particular condition capable of being found by one other than the patient. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after a condition manifests in a subject, while “prophylactic” treatment is initiated before a condition manifests in a subject. Prophylactic treatment may be administered to a subject at risk of having a condition. “At risk” refers to a subject that may or may not actually possess the described risk. Thus, for example, a subject “at risk” of infection by a microbe is a subject present in an area where individuals have been identified as infected by the microbe and/or is likely to be exposed to the microbe even if the subject has not yet manifested any detectable indication of infection by the microbe and regardless of whether the subject may harbor a subclinical amount of the microbe. In the case of a non-infectious condition, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition.

The protein scaffolds described herein may have utility in molecular imaging applications including, for example, both traditional molecular imaging techniques (e.g., magnetic resonance imaging (MRI), positron emission tomography (PET), single photon emission computed tomography (SPECT), ultrasound, photoacoustic, and fluorescence) and microscopy and/or nanoscopy imaging techniques (e.g., total internal reflection fluorescence (TIRF)-microscopy, stimulated emission depletion (STRED)-nanoscopy, and atomic force microscopy (AFM).

The protein scaffolds described herein have in vitro and in vivo detection, diagnostic, and/or therapeutic utilities. For example, a protein scaffold may be included in a detection composition for use in a detection method. The method generally can include allowing a protein scaffold that specifically binds to a target of interest with a sample that includes the target of interest, then detecting the formation of a protein scaffold:target complex. Thus, the protein scaffold may be designed to include a detectable marker such as, for example, a radioactive isotope, a fluorescent marker, an enzyme, or a colorimetric marker. As another example, the protein scaffolds described herein can be administered to cells in culture, e.g. in vitro or ex vivo, or in a subject, e.g., in vivo, to treat—either therapeutically or prophylactically—or diagnose a variety of disorders.

This disclosure further provides the use of the scaffolds described herein for prophylaxis, diagnosis, management, treatment, or amelioration of one or more symptoms and/or clinical signs associated with diseases or disorders including, but not limited to, cancer, inflammatory and autoimmune diseases, infectious diseases, either alone or in combination with other therapies.

Moreover, many cell surface receptors activate or deactivate as a consequence of crosslinking of subunits. The protein scaffolds described herein may be used to stimulate or inhibit a response in a target cell by crosslinking of cell surface receptors. In another embodiment, a protein scaffold as described herein may be used to block the interaction of multiple cell surface receptors with antigens. In another embodiment, a protein scaffold as described herein may be used to strengthen the interaction of multiple cell surface receptors with antigens. In another embodiment, it may be possible to crosslink a homodimer and/or heterodimer of a cell surface receptor using a protein scaffold as described herein that includes binding domains that share specificity for the same antigen, or bind two different antigens. In another embodiment, a protein scaffold as described herein could be used to deliver a ligand, or ligand analogue to a specific cell surface receptor.

The disclosure further provides methods of targeting epitopes not easily accomplished with traditional antibodies. For example, in one embodiment, a protein scaffold as described herein may be used to first target an adjacent antigen and while binding, another binding domain may engage the cryptic antigen.

This disclosure also provides methods of using a protein scaffold to bring together distinct cell types. In one embodiment, a protein scaffold as described herein may bind a target cell with one binding domain and recruit another cell via another binding domain. In another embodiment, the first cell may be a cancer cell and the second cell is an immune effector cell such as an NK cell. In another embodiment, a protein scaffold as described herein may be used to strengthen the interaction between two distinct cells, such as an antigen presenting cell and a T cell to possibly boost the immune response.

This disclosure also provides methods of using scaffolds proteins to ameliorate or treat, either prophylactically or therapeutically, cancer or a symptom or clinical sign thereof. In various embodiments, the methods may be useful in the treatment of cancers of the head, neck, eye, mouth, throat, esophagus, chest, skin, bone, lung, colon, rectum, colorectal, stomach, spleen, kidney, skeletal muscle, subcutaneous tissue, metastatic melanoma, endometrial, prostate, breast, ovaries, testicles, thyroid, blood, lymph nodes, kidney, liver, pancreas, brain, or central nervous system.

As used herein, the term “and/or” “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Materials and Methods

### Library Construction

Oligonucleotides, including amino acid and loop length diversity, were synthesized by IDT DNA Technologies (sequences in Supplementary Tables 1-2). Full-length Fn3HP amplicons were assembled by overlap extension PCR. The library of pooled diversified DNA was homologously recombined into a pCT yeast surface display vector (Lipovsek et al., 2007, J Mol Biol. 368(4):1024-1041) within yeast strain EBY100 (Boder E T, Wittrup K D, 1997, Nat Biotechnol. 15(6):553-557) during electroporation transformation. The protocol was similar to that previously described (Benatuil et al., 2010, Protein Eng Des Sel. 23(4):155-159). Yeast at OD₆₀₀=1.3-1.5 were washed twice with cold water and once with buffer E (1 M sorbitol, 1 mM CaCl₂) and resuspended in 0.1 M lithium acetate, 10 mM Tris, 1 mM ethylenediaminetetraacetic acid, pH 7.5. Fresh dithiothreitol was added to 10 mM. Cells were incubated at 30° C., 250 rpm for 30 minutes. Cells were washed thrice with cold buffer E and resuspended to 1.4 billion cells per 0.3 mL buffer E. Six μg of linearized pCT vector (Hackel et al., 2010, J Mol Biol. 401(1):84-96) and 200 pmol of ethanol precipitated gene insert were added and transferred to a 2-mm cuvette. Cells were electroporated at 1.2 kV and 25 diluted in YPD (10 g/L yeast extract, 20 g/L peptone, 20 g/L dextrose), and incubated at 30° C. for one hour. Cells were pelleted and resuspended in 100 mL SD-CAA (16.8 g/L sodium citrate dehydrate, 3.9 g/L citric acid, 20.0 g/L dextrose, 6.7 g/L yeast nitrogen base, 5.0 g/L casamino acids). Plasmid-containing yeast were quantified by dilution plating on SD-CAA agar plates. The construction of generation two DNA was done in the same manner.

Each resulting Fn3HP naïve yeast library was evaluated for proper library construction by Sanger sequencing clonal plasmids harvested from the transformed yeast (57 clones from generation one and 15 from generation two naive libraries). The yeast libraries were also labeled with biotinylated anti-HA antibody and anti-c-myc antibody (9E10, Covance Antibody Products, BioLegend, Inc., San Diego, Calif.) to detect the presence of N-terminal and C-terminal epitopes present on either side of the Fn3HP clones, respectively, via flow cytometry. The fractional detection of cells displaying both HA and c-myc, compared to those displaying HA alone, is indicative of full-length, stop codon-free clones.

### Binder Maturation and Evolution

The Fn3HP yeast library was grown in SD-CAA selection media for several doublings (about 20 h) in an incubator shaker at 30° C. until an OD₆₀₀ value of 6.0 was reached, at which time the yeast were centrifuged and resuspended in SG-CAA induction media (10.2 g/L Na₂HPO₄.7H₂O, 8.6 g/L NaH₂PO₄.H₂O, 19.0 g/L galactose, 1.0 g/L dextrose, 6.7 g/L yeast nitrogen base, 5.0 g/L casamino acids) and grown overnight. The induced library was sorted twice via multivalent magnetic bead selections (Ackerman et al., 2009, Biotechnol Prog. 25(3):774-783) via depletion of non-specific binders on avidin-coated beads and control protein-coated beads followed by enrichment of specific binders on target-coated beads. The pair of magnetic sorts was followed by a flow cytometry selection for full-length clones using the 9E10 antibody against the C-terminal c-myc epitope tag. Genes were mutated via error-prone PCR with loop shuffling (Hackel et al., 2008, J Mol Biol. 381(5):1238-1252), then electroporated into yeast (EBY100) as previously described (Benatuil et al., 2010, Protein Eng Des Sel. 23(4):155-159). Target binding populations were isolated when at least one of two criteria was achieved: (i) magnetic bead sorting enrichment of target binding population was at least ten-fold greater than both avidin binding and non-specific control binding, and/or (ii) cytometry analysis at target concentration of 50 nM revealed signal above background. Clones meeting these criteria are referred to as mid- and high- affinity binders, respectively, within this document.

### Illumina MiSeq Sample Preparation and Sequence Analysis

Plasmid DNA was isolated from protein-displaying yeast using ZYMOPREP Yeast Plasmid Miniprep II (Zymo Research Corp., Irvine, Calif.). DNA samples were divided into separate groups based on library generation of origin and binding affinity. Three categories were included for each generation: naive clones from the initial libraries, mid-affinity binders collected via magnetic bead sorting, and high-affinity binders collected using FACS. In total, six pools of DNA were isolated and uniquely analyzed in association with generations 1 and 2. Following plasmid DNA extraction, two rounds of PCR were completed to assemble the Fn3HP gene fragment with Illumina primers, index tags, multiplexing bar codes, and TRUSEQ universal adapter (Illumina, Inc., San Diego, Calif.). For all PCR conducted during amplicon library preparation, KAPA HiFi polymerase was used as it has been shown to reduce clonal amplification bias due to GC content as well as fragment length bias. Compatible multiplexing and adapter primers were designed according to TRUSEQ (Illumina, Inc., San Diego, Calif.) sample preparation guidelines. Amplicons were pooled and supplemented with 25% PhiX control library to increase MISEQ (Illumina, Inc., San Diego, Calif.) read accuracy. Illumina MISEQ paired-end sequencing with 2×250 read length was conducted (University of Minnesota Genomics Center) to obtain 7.2×10⁶ pass filter (PF) reads from the populations of interest, of which 90% of all pass filter bases were above Q30 quality metric (99.9% read accuracy).

### Sequence Analysis

Raw data generated through MISEQ (Illumina, Inc., San Diego, Calif.) consisted of twelve files formatted as FASTQ. A forward and reverse read file was generated for each of the six multiplexed sublibraries. Assembly of paired end reads was done using PANDAseq (Masella et al., 2012, BMC Bioinformatics. 13(1):31). Assembled reads were analyzed using in-house Python code (Sanner M, 1999, J Mol Graph Model. 17:57-61). Analysis work flow for each of the six subgroups (e.g. naive, mid-affinity, high-affinity populations originating from first and second generation libraries) consisted of first identifying full-length fibronectin DNA sequences, isolating each of the three diversified loop regions, and, lastly, calculating the amino acid frequency at each site. Additional calculations were necessary for the mid-affinity and high-affinity populations to both remove statistically rare events and avoid overcounting dominant clones. The removal of background (i.e,. singleton and doublet sequences) was a precaution taken when analyzing the mid-affinity populations to account for the rare non-binding clones inherently collected via magnetic bead sorting. To address the potential detriment of overcounting within all binding populations, the sequences for each loop region were clustered based on 80% or greater sequence homology. For each cluster of similar sequences, the summation of the amino acids at each site were weighted by a power of one-half, then aggregated across all clusters. The resulting weighted sitewise amino acid values were used for frequency calculations. Statistical analysis was performed using two sample student's t-test. Statistical significance was assessed while adjusting for familywise error rate using Bonferroni method, denoted at level α=0.005.

### Stability Assessment

High-affinity clones from three separate target binding campaigns of the current study were individually evaluated for stability using thermal denaturation midpoint, T, in the context of yeast surface display, as previously described (Hackel et al., 2008, J Mol Biol. 381(5):1238-1252). Seven random clones from the second generation initial library were produced with a C-terminal six-histidine tag in BL21(DE3) and purified by immobilized metal affinity chromatography and reverse phase high performance liquid chromatography. Purified proteins (1 mg/mL in 2 mM 4-(2-Hydroxyethyl)piperazine-1-ethanesulfonic acid, 50 mM NaCl, 2 mM ethylenediaminetetraacetic acid, 1 mM dithiothreitol) analyzed via circular dichroism using a JASCO J815 instrument. Measurements of molar ellipticity were taken at 218 nm while heating from 20° C.-98° C. at a rate of 1° C./minute. Wild-type Fn3HP was analyzed in the same fashion. Stability measurements of 15 engineered fibronectin clones were retrieved from previously published studies wherein library design was implemented through a binary approach: broadly diversifying the anticipated paratope, using NNS (Xu et al., 2002, Chem Biol. 9(8):933-942) and NNB (Hackel et al., 2008, J Mol Biol. 381(5):1238-1252) codons, and fully conserving all other positions.

### Quantitative Parameterization to Guide Library Design

A sitewise amino acid diversity matrix (D*) is calculated from Equation 1:

$${D}^{*} = {\sum }_{k}^{a , b , c} ( {α}_{k} + {β}_{k} \cdot ɛ ) \cdot {D}_{k} \qquad ( Eq . 1 )$$

where αₖ and βₖ are tunable weights to scale the primary input data as a function of exposure score, ε. The site-specific exposure score is calculated as the product of the solvent exposed surface area (Hackel et al., 2010 J Mol Biol. 401(1):84-96) and relative exposure to target binding interface. Dₖ is the sitewise amino acid frequency distribution associated with each of the three primary input data sets (theoretical stability, natural sequence frequency in homologs, and complementarity).

The model was optimized using a least-square method to minimize error between the calculated matrix, D*, and objective matrix, defined as the sitewise amino acid diversity matrix generated from the second generation library binder sequences. Constraints are placed such that each set of a values sum to 1.0 and each set of β values sum to zero.

### Stability Matrices

FoldX (Schymkowitz et al., 2005, Nucleic Acids Res. 33(Web Server issue):W382-W388) was used to determine the mutability of commonly diversified sites within the tenth type III domain of human fibronectin in the context of several structures cataloged on the Protein Data Bank (PDBs: 1FNA, 1TTG, 2OBG, 2OCF, 2QBW, 3CSB, 3CSG, 3K2M, 3QHT, 3RZW, 3UYO; Berman et al., 2000, The Protein Data Bank. 28(1):235-242). After performing FoldX repair, a collection of at least fifty random mutants were generated for each of the eleven structures by randomizing the BC, DE, and FG loop regions in accordance with the second generation diversity design scheme. At this point, baseline stabilities were individually calculated for each mutant. To analyze the stability impact upon residue substitution for each position in the diversified regions, all 19 natural residue substitutions were individually introduced to the random mutants. The change in stability (ΔΔG_{folding}) upon mutation was then calculated for each PDB structure's collection of mutants. At each site, the stability impact upon substitution to each amino acid was calculated, creating stability matrices for each starting PDB. The sequences corresponding to the wild-type structures were aligned to account for loop length diversity. Average ΔΔG_{folding} values were calculated for all 20 amino acids at each diversified site.

### Target Exposure

The likelihood of a loop position to be proximal to or directly involved with a target binding interface is influenced both by exterior exposure of the side chain (i.e., solvent accessible surface area) as well as its proximity to a region offering sufficient surface area to enable the required enthalpic interactions. The latter was quantified here on a sitewise basis across eleven Fn3 crystal structures. To calculate this metric, each structure was loaded into PyMOL (DeLano W L, 2002, CCP4 Newsletter On Protein Crystallography 40:82-92), each BC,

DE, and FG loop site was mutated to alanine, and then each loop site was colored white (all other sites were black). To identify the ideal angles to view the maximum interfacial area, tools were developed using Python (Sanner M, 1999. J Mol Graph Model. 17:57-61) to analyze white pixel counts at the current viewpoint and translate to A² using a scalar atom. A coarse-grain rotational search for this ideal angle was completed followed by fine-grain angle optimization. With this maximally exposed view of the paratope, one can screen for additional angles with 95% of the maximal surface area. With the set of angles greater than this threshold, one can highlight individual residues and look for the angle of maximum exposure and its respective exposure area value. Each starting PDB file had its residues normalized to a maximum score of one, then averaged across all files.

TABLE 1
Amino acid diversity encoded in first generation library. Each row was constructed as a separate sublibrary. CDR′ refers to
a degenerate codon with the following nucleotide frequencies: 20% A, 15% C, 25% G, and 40% T at site 1, 50% A, 25% C, 15% G, and
10% T at site 2, and 0% A, 45% C, 10% G, and 45% T at site 3. Loop length diversity was afforded at mid-loop positions by inserting
CDR′ diversity sites between sites P25-V29 and G79-S85 of the BC and FG loop, respectively, as denoted by subscripts within
table. Length diversity at the DE loop occurred between S53-S55, consisting of diversity matching that of K54 as shown below.

| | BC Loop | | | | | | | | |
|---|---|---|---|---|---|---|---|---|---|
| | | | | AVT | | | | DE Loop | |
| Site | D23 | A24 | P25 | 26-28 | V29 | R30 | Y31 | G52 | S53 |
| Sublib. 1 | D | A | P | CDR′₁₋₄ | AST | CDR′ | G | G | S |
| Sublib. 2 | DSYA | AS | PS | CDR′₁₋₄ | AST | CDR′ | GS | GS | S |
| Sublib. 3 | DSYA | ASYD | PSYH | CDR′₁₋₄ | AST | CDR′ | GSYCDN | GSYCDN | SYNT |
| Sublib. 4 | ACDGNSTY | ACDGNSTY | ACDGNSTY | CDR′₁₋₄ | AST | CDR′ | ACDGNSTY | ACDGNSTY | ACDGNSTY |
| Sublib. 5 | CDR′ | CDR′ | CDR′ | CDR′₁₋₄ | AST | CDR′ | CDR′ | CDR′ | CDR′ |

| | | | | FG Loop | | | | | |
|---|---|---|---|---|---|---|---|---|---|
| | DE Loop | | | | GR | | DSPAS | | |
| Site | K54 | S55 | T56 | T76 | 77-78 | G79 | 80-84 | S85 | K86 |
| Sublib. 1 | N₀₋₂ | S | T | T | CDR′ | GDNSYC | CDR′₁₋₅ | S | N |
| Sublib. 2 | (NS)₀₋₂ | S | TS | TS | CDR′ | GDNSYC | CDR′₁₋₅ | S | NS |
| Sublib. 3 | (NSYT)₀₋₂ | SYNT | TSYN | TSYN | CDR′ | GDNSYC | CDR′₁₋₅ | SYNT | NSYT |
| Sublib. 4 | (ACDGNSTY)₀₋₂ | ACDGNSTY | ACDGNSTY | ACDGNSTY | CDR′ | GDNSYC | CDR′₁₋₅ | ACDGNSTY | ACDGNSTY |
| Sublib. 5 | CDR′₀₋₂ | CDR′ | CDR′ | CDR′ | CDR′ | GDNSYC | CDR′₁₋₅ | CDR′ | CDR′ |

TABLE 2
Amino acid diversity encoded in second generation library. CDR′ refers to a degenerate codon
with the following nucleotide frequencies: 20% A, 15% C, 25% G, and 40% T at site 1, 50% A, 25% C,
15% G, and 10% T at site 2, and 0% A, 45% C, 10% G, and 45% T at site 3. Loop length diversity was
afforded at mid-loop positions by inserting CDR′ diversity sites between sites P25-V29 and G79-
S85 of the BC and FG loop, respectively, as denoted by subscripts within table. Length diversity at
the DE loop occurred between S53-S55, consisting of either wild-type length or the exclusion of K54.

| | BC Loop | | | | | | | | |
|---|---|---|---|---|---|---|---|---|---|
| | | | | AVT | | | | DE Loop | |
| Site | D23 | A24 | P25 | 26-28 | V29 | R30 | Y31 | G52 | S53 |
| Generation 2 | D | A/ASYDNT | P/PSYH | CDR′₂₋₄ | AST | CDR′ | GY | G | SYNT |

| | | | | FG Loop | | | | | | |
|---|---|---|---|---|---|---|---|---|---|---|
| | DE Loop | | | | | | | DSPAS | | |
| Site | K54 | S55 | T56 | T76 | G77 | R78 | G79 | 80-84 | S85 | K86 |
| Generation 2 | (NSYT)₀₋₁ | SYNT | TSYN | TSGA | GSYADCNT | CDR′ | GSDN | CDR′₁₋₅ | S | N |

TABLE 3
Illumina sequencing statistics for 251 basepair paired end run on
MISEQ for six uniquely barcoded libraries. Collectively, 7.2 million
pass filter reads were obtained, including 25% PhiX control library.
Within all pass filter read bases, 90% were above Q30 quality metric
(99.9% read accuracy). Thorough sampling was observed across all
six libraries with a coefficient of variance of 30%.

| | Generation 1 | | Generation 2 | |
|---|---|---|---|---|
| | Bead | Flow | Bead | Flow |
| Total Sequences | 181,716 | 239,934 | 259,519 | 225,205 |
| Unique Sequences | 65,971 | 47,957 | 128,485 | 103,615 |
| Unique Families | 3,334 | 256 | 3,733 | 1,234 |

TABLE 4
Enrichment of framework mutations. Full length fibronectin sequences
from second generation naïve and binder populations were
analyzed. Five framework positions demonstrated enrichment for non-
wild type residues. Prevalence of these amino acids in natural homologs
is shown in the two right most columns. Bottom row indicates median
values of wild-type and any single mutant across all sites.

| | Frequency in this work | | Natural Frequency | |
|---|---|---|---|---|
| Mutation | Initial | Binders | Wild-type | Mutant |
| S2P | 0.1% | 5% | 28% | 14% |
| I20V | 0.5% | 4% | 16% | 30% |
| S43F | 1% | 9% | 19% | 1% |
| P44S | 11% | 34% | 23% | 7% |
| I88T | 0.2% | 10% | 13% | 3% |
| median(all sites) | 0.01% | <0.01% | 21% | 2% |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ NO: 1
VSDVPRDLEV VAATPTSLLI SWHHLSCSDS RYYRITYGET
GGNSPVQEFT VPGSYDSYSA TISGLKPGVD YTATVYAVTR
RGYDCPHPIS INYRTEIDKP SR
SEQ NO: 2
ASDVPRDLEV IAATPTSLLI SWLPSYSPGY YRITYGETGG
NSPVQEFTVP GSYDSYSATI SGLKPGVDYT ITVYAVTGRV
SSYPTSINCR TGIDKPSR
SEQ NO: 3
SSDSPRNLEV TNATPNSLTI SWDDPYPAHA HYYRITYGET
GGNSSSQEFT VPGNYYYATI SGLKPGQDYT ITVYAVGTNS
YSNPISINYR TETDKPSQ
SEQ NO: 4
SPDSPRNLEV TNATPNSLTI SWDDPYPAHA HYYRITYGET
GGNSSSQEFT VPGNYYYATI SGLKPGQDYT ITVYAVGCKN
CYSNPISINY RTEIDRPSQ
SEQ NO: 5
SSDSPRNLEV TNATPNSLTI SWDDPYPTHA HYYRITYGET
GGNSSSQEFT VPGNYYYATI SGLKPGQDYT ITVYAVGSHG
HYSNPTSINY RTEIDKPSQ
SEQ NO: 6
SSDSPRNLEV TNATPNSLTI SWDDPYPAHA HYYRITYGET
GGNSSSQEFT VPGNYYYATI SGLKPGQDYT ITVYAVTDHN
YSYSNPISIN CRTEIDKPSQ
SEQ NO: 7
SSDSPRNLEV TNATPNSLTV SWDDPYPTHA HYYRITYGET
GGSSSSQEFT VPGNYYYATI SGLKPGQDYT ITVYAVSNDN
SSSNPISINY RTEIDKPSQ
SEQ NO: 8
SSDSPRNLEV TNATPNSLTI SWDAPPALDS SGYRITYSET
GGNSPSQEFT VPGNYYYATI SRLKPGQDYT ITVYTVSHHD
RYGPYSNP
SEQ NO: 9
SSDSPRDLEV TNATPNSLTI SWDAPPALDS SGYRITYGET
GGNSPSQEFT VPGNYYYATI SGLKPGQDYT ITVYAVADNS
HNPNPISINY RTEIDKPSQ
SEQ NO: 10
SSDSPRNLEV INATPNSLTI SWDAPPALDS SGYRITYGET
GGNSPSQEFT VPGNYYYATI SGLRPGQDYT ITVYAVSNID
NSNPVSINYR TEIDKPSQ

What is claimed is:

1. A non-naturally occurring protein scaffold comprising:
a frame comprising a plurality of structural domains comprising at least one f3 structure or at least one a helix; and
at least one loop region that specifically binds hepatocyte growth factor receptor (MET) and comprises an amino acid sequence that varies from a naturally-occurring loop region by at least one amino acid deletion, amino acid substitution, or amino acid addition.

2. The protein scaffold of claim 1 wherein the frame is derived from fibronectin.

3. The protein scaffold of claim 1 or claim 2 wherein the at least one loop region comprises at least one of SEQ ID NO:1-10.

4. A pharmaceutical composition comprising the protein scaffold of any preceding claim.

5. A detection composition comprising the protein scaffold of any one of claims 1-4.

6. The detection composition of claim 5 further comprising a detectable marker.

7. The detection composition of claim 6 wherein the detectable marker comprises a radioactive isotope, a fluorescent marker, or a colorimetric marker.

8. A method for detecting hepatocyte growth factor receptor (MET), the method comprising:
providing a protein scaffold of any one of claims 1-3 that specifically binds MET;
contacting the protein scaffold with a sample that includes MET; and
detecting at least one protein scaffold:target molecule complex.

9. A method comprising:
administering an effective amount of a pharmaceutical composition of claim 4 to a subject in need of treatment for a condition treatable by the pharmaceutical composition.

10. The use of a protein scaffold of any one of claims 1-3 in the manufacture of a pharmaceutical composition.
